(19) United States
(12) Patent Application Publication
(10) Pub. No.: US 2025/0308664 A1
(43) Pub. Date: Oct. 2, 2025

(54) CLINICAL TRIAL PLANNING DEVICE AND CLINICAL TRIAL PLANNING METHOD

(71) Applicant: Hitachi, Ltd., Tokyo (JP)

(72) Inventors: Yutaro KIDO, Tokyo (JP); Kunihiko KIDO, Tokyo (JP); Hiroko OTAKI, Tokyo (JP); Yoichi NAKAMOTO, Tokyo (JP)

(73) Assignee: Hitachi, Ltd., Tokyo (JP)

(21) Appl. No.: 19/052,957

(22) Filed: Feb. 13, 2025

(30) Foreign Application Priority Data
Mar. 26, 2024 (JP) 2024-050371

(51) Int. Cl. G16H 20/10; G06Q 30/0202; G16H 10/20

(52) U.S. Cl. CPC G16H 20/10; G06Q 30/0202; G16H 10/20

(57) ABSTRACT

This invention provides a method capable of realizing an efficient clinical trial in which development situations of drugs of competitor companies are considered. In a preferable aspect of the present invention, a clinical trial planning device is provided which includes a clinical trial planning unit. The clinical trial planning unit includes a development progress degree calculating unit calculating a development progress degree of a second drug of which patient indication corresponds to that of a first drug for which a clinical trial is to be planned, on the basis of a competitive drug design indicating a situation of a clinical trial related to the second drug, and generates a clinical trial plan obtained by optimizing a period of a clinical trial of the first drug and expected sales of the first drug on the basis of the development progress degree.

### BACKGROUND OF THE INVENTION

The present invention relates to a technique of optimizing planning of a clinical trial.

As the number of cancer patients rapidly increases due to aging of society, competition of development of drugs of cancers is becoming fiercer. Since segmentation of indications of drugs of cancers proceeds with advancement of molecular diagnosis technology and the number of clinical trials increases, it is becoming difficult to choose an indication of a profitable drug in development competition with other companies.

A pharmaceutical company determines a trial design and an indication so that a drug which can be differentiated in indications and effects from drugs of other companies is marketed ahead of the other companies while seeing process of clinical trials of the competitor companies.

The plan is re-examined on the basis of quarterly information of the societies. It is, however, difficult to make a plan which increases the pharmaceutical price in short time due to information overload and manpower shortage, and it causes delay in development and increase in costs.

To solve the problem, quantitative clinical trial planning methods are developed (for example, Japanese Unexamined Patent Application Publication (Translation of PCT Application) Nos. 2021-533453 and 2005-527921). As typical methods, clinical trial planning methods of calculating a trial design of each trial process so as to maximize profit are examined (for example, Wiklund S. J. (2019) “A modelling framework for improved design and decision-making in drug development”, PLOS ONE, 14(8), e0220812, https://doi.org//10.1371/journal.pone.0220812, and Parke, T., Marchenko, O., Anisimov, V., Ivanova, A., Jennison, C., Perevozskaya, I., and Song, G. (2017) “Comparing oncology clinical programs by use of innovative designs and expected net present value optimization: Which adaptive approach leads to the best result?”, Journal of biopharmaceutical statistics, 27(3), 457-476, https://doi.org/10.1080/10543406.2017.1289949.

### SUMMARY OF THE INVENTION

Japanese Unexamined Patent Application Publication (Translation of PCT Application) No. 2021-533453 discloses a method and system for making a clinical trial executing plan, concretely, for making a criterion of inclusion/exclusion which is used to determine a patient population as a target. Japanese Unexamined Patent Application Publication (Translation of PCT Application) No. 2005-527921 discloses a system for predicting and chasing a substance in a clinical trial.

In Wiklund S. J. (2019) “A modelling framework for improved design and decision-making in drug development”, PLOS ONE, 14(8), e0220812, https://doi.org//10.1371/journal.pone.0220812, and Parke, T., Marchenko, O., Anisimov, V., Ivanova, A., Jennison, C., Perevozskaya, I., and Song, G. (2017) “Comparing oncology clinical programs by use of innovative designs and expected net present value optimization: Which adaptive approach leads to the best result?”, Journal of biopharmaceutical statistics, 27(3), 457-476, https://doi.org/10.1080/10543406.2017.1289949, profit (net present value) is calculated from the cost and expected sales of a clinical trial of a development drug estimated from candidates of trial designs, and a trial design which maximizes the profit is obtained.

However, those methods are planning methods using information of only a drug to be developed, and development situations of competitor companies are not considered. As a result, there is a risk that when a competitor company puts a drug of the same indication on the markets first, differentiation from the drug cannot be done, and profit cannot be obtained. Particularly, in cancers for which the number of clinical trials is large, since many pharmaceutical companies develop drugs in short time, such a risk is high. To increase the profit in development of a cancer drug, a method in which the development situation of a drug of a competitor company is considered is required.

Consequently, an object of the present invention is to provide a method capable of realizing an efficient clinical trial in which development situations of drugs of competitor companies are considered.

In a preferable aspect of the present invention, a clinical trial planning device is provided which includes a clinical trial planning unit. The clinical trial planning unit includes a development progress degree calculating unit calculating a development progress degree of a second drug of which patient indication corresponds to that of a first drug for which a clinical trial is to be planned, on the basis of a competitive drug design indicating a situation of a clinical trial related to the second drug, and generates a clinical trial plan obtained by optimizing a period of a clinical trial of the first drug and expected sales of the first drug on the basis of the development progress degree.

More specifically, the clinical trial plan is a combination of a plurality of trial designs related to the first drug.

In another preferable aspect of the present invention, a clinical trial planning method is provided which is executed by an information processing device having a control unit, a clinical trial planning unit, a storage unit, a display unit, and an input unit. The clinical trial planning unit executes: a first step of reading a plurality of trial designs defining a patient indication and the number of samples, related to a first drug; a second step of reading a plurality of competitive drug designs defining a patient indication and a trial phase, related to a second drug; a third step of specifying a competitive drug design having the same patient indication from the competitive drug designs for each of the trial designs; a fourth step of calculating a development progress degree on the basis of the trial phase of the competitive drug design specified for each of the trial designs; a fifth step of calculating a trial period on the basis of the number of samples for each of the trial designs; a sixth step of calculating expected sales on the basis of a market scale and the development progress degree for each of the trial designs; and a seventh step of generating a clinical trial plan made by a combination of the trial designs by using a reward function having the trial period and the expected sales as variables, for a combination of the trial designs allocated to trial processes of the number which is preliminarily determined.

More specifically, in the seventh step, an optimum plan model generated by reinforcement learning using miniaturization of the total sum of the trial period and maximization of the total sum of the expected sales as rewards is used.

It is possible to provide a method capable of realizing an efficient clinical trial in which development situations of drugs of competitor companies are considered.

### BRIEF DESCRIPTION OF THE DRAWINGS

FIG. 1A is a conceptual diagram explaining clinical trial planning as an embodiment of the present invention.

FIG. 1B is a conceptual diagram explaining trial designs as an embodiment of the present invention.

FIG. 1C is a table illustrating a list of trial processes in a clinical trial plan as an embodiment of the present invention.

FIG. 2 is a block diagram illustrating a schematic configuration of a clinical trial planning device according to an embodiment of the present invention.

FIG. 3 is a flowchart for explaining a clinical trial planning process executed by the clinical trial planning device according to the embodiment of the present invention.

FIG. 4 is a flowchart for explaining a development progress degree calculating process executed by the clinical trial planning device according to the embodiment of the present invention.

FIG. 5 is a flowchart for explaining a trial period calculating process executed by the clinical trial planning device according to the embodiment of the present invention.

FIG. 6 is a flowchart for explaining an expected-sales calculating process executed by the clinical trial planning device according to the embodiment of the present invention.

FIG. 7 is a flowchart for explaining an optimum plan model learning process executed by the clinical trial planning device according to the embodiment of the present invention.

FIG. 8 is a flowchart for explaining an optimum plan estimating process executed by the clinical trial planning device according to the embodiment of the present invention.

FIG. 9 is a table illustrating the data structure of a trial design held by the clinical trial planning device according to the embodiment of the present invention.

FIG. 10 is a table illustrating the data structure of a competitive drug design held by the clinical trial planning device according to the embodiment of the present invention.

FIG. 11 is a table illustrating the data structure of a development progress degree held by the clinical trial planning device according to the embodiment of the present invention.

FIG. 12 is a table illustrating the data structure of a trial period held by the clinical trial planning device according to the embodiment of the present invention.

FIG. 13 is a table illustrating the data structure of a market scale held by the clinical trial planning device according to the embodiment of the present invention.

FIG. 14 is a table illustrating the data structure of expected sales held by the clinical trial planning device according to the embodiment of the present invention.

FIG. 15 is a table illustrating the data structure of an optimum plan held by the clinical trial planning device according to the embodiment of the present invention.

FIG. 16 is an explanatory diagram illustrating a display example of a one's company/competitor drug trial design input screen by the clinical trial planning device according to the embodiment of the present invention.

FIG. 17 is an explanatory diagram illustrating a display example of a development progress degree calculating screen by the clinical trial planning device according to the embodiment of the present invention.

FIG. 18 is an explanatory diagram illustrating a display example of an optimum plan display screen by the clinical trial planning device according to the embodiment of the present invention.

### DETAILED DESCRIPTION OF THE PREFERRED EMBODIMENTS

Embodiments will be described in detail with reference to the drawings. The present invention, however, is not interpreted by being limited to the following description of the embodiments. A person skilled in the art will easily understand that a concrete configuration can be changed without departing from the idea or gist of the present invention.

In the configuration of the following embodiments, the same reference numeral will be used to the same part or a part having a similar function commonly in different drawings, and repetitive description may be omitted.

In the case where there are a plurality of elements having the same or similar function, description may be given by attaching different suffixes to the same sign. When it is unnecessary to distinguish a plurality of elements, description may be given without suffixes.

Writing such as “the first”, “the second”, and “the third” in the specification is to identify components and does not always limit the number, order, or content. A number for identifying a component is used by context, and a number used in a context does not always refer to the same configuration in another context. A component identified by a certain number is not hindered from also having the function of a component identified by another number.

The position, size, shape, range, and the like of each of the configurations illustrated in the diagrams and the like may not express the actual position, size, shape, range, and the like in order to make the present invention easily understood. Therefore, the present invention is not limited to the positions, sizes, shapes, ranges, and the like disclosed in the diagrams and the like.

The publications, patents, and patent applications cited in the specification constitute a part of the description of the specification.

A component expressed in a singular form in the specification includes a plural form unless otherwise clearly specified in a context.

In the embodiment, a method of planning a clinical trial which optimizes a trial design by using a development progress degree of a competitor company is developed.

Planning of a clinical trial in the embodiment is a method of achieving two goals. The first is to maximize expected sales estimated in consideration of a development progress degree calculated by using a development phase of a drug of the same indication as that of a competitor company in order to be differentiated from the competitor company in the indication. The other is to minimize a trial period so as to market a drug ahead of other companies.

For high expected sales, it is necessary to indicate efficacy of a drug at a high target response rate by increasing the number of samples in a design of a clinical trial. On the other hand, when the number of samples is increased, the trial period becomes longer. To achieve two goals in tradeoff between the expected sales and the trial period, the embodiment is characterized by obtaining an optimum plan by a reinforcement learning method using, as a reward function, a mathematical formula which evaluates expected sales and a trial period calculated from a design of a clinical trial.

According to the above-described configuration, by developing a drug of large expected-sales in a short trial period, expected revenue of a drug development can be increased. When expected sales is high, sales which can be expected in drug development is increased. A short trial period leads to higher probability of obtaining sales by marketing a drug ahead of other companies and reduction in the cost. Particularly, it is effective in diseases such as cancer in which development competition with other companies is fierce and drugs of many indications are developed in a short period.

### First Embodiment

Hereinafter, a clinical trial planning device in a preferred embodiment of the present invention will be described in detail with reference to the drawings.

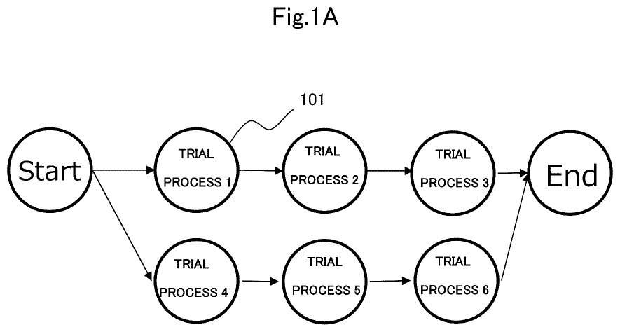

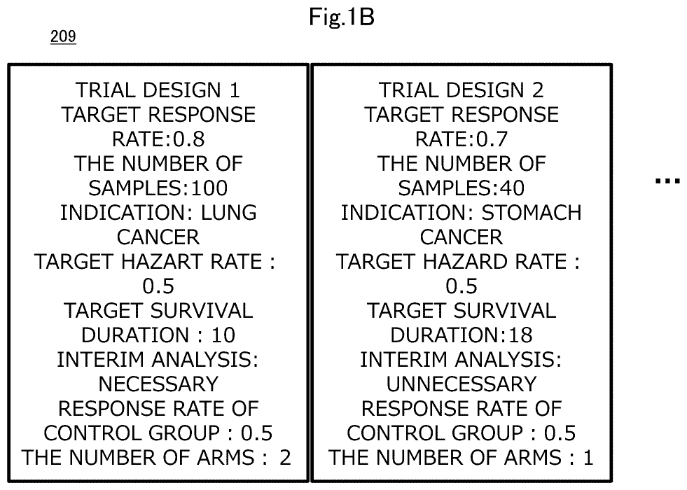

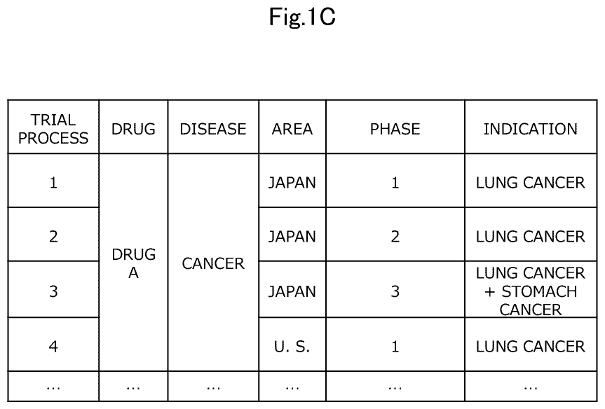

FIG. 1A is a diagram explaining clinical trial planning as an embodiment of the present invention. The target of the clinical trial planning is one drug (example: drug A) for one disease (example: cancer). Trial processes 101 are obtained by dividing the clinical trial planning on the basis of areas and trial phases in which a trial is executed. That is, one common drug corresponds to the trial processes.

In the embodiment to be described, allocation of any of trial designs which will be described hereinafter to each of the trial processes 101 of the clinical trial will be called planning of the clinical trial.

FIG. 1B is a conceptual diagram illustrating an example of a trial design 209 as an embodiment of the present invention. Name or code (example: trial design 1) uniquely specifying a trial design, a target response rate (example: 0.8 or 80% or the like), the number of samples (subjects) (example: 100), indication (example: lung cancer), target hazard rate (0.5), target survival duration (example: 10 years), interim analysis (example: necessary or unnecessary), response rate of control group (example: 0.5 or 50% or the like), the number of arms (example: 2), and the like are included. A part of the above may be omitted. Other data indicating a target, a characteristic, and a specification of a development drug may be included.

The indication refers to a disease or symptom as an object of use of the drug. In the embodiment, it is assumed that information of the indication is necessary. With respect to the hazard rate, in a method of objectively comparing relative risk degrees used in a clinical trial or the like, when a new drug A which is desired to be examined in a clinical trial and a comparable drug B are compared and the result is that the hazard rate in the clinical trial is 0.5, it means that the drug A decreased the risk by 50% as compared with the drug B. The number of arms indicates “the number of control groups+1”.

Many pieces of data as described above are commonly used in the pharmaceutical industry, and data in databases or the like has similar data formats.

FIG. 1C indicates a list of trial processes in a clinical trial plan. This clinical trial plan relates to “drug A”, and a target disease is “cancer”. For example, in trial process 1,the area in which a trial is implemented is “Japan”, the phase is “1”, and indication is “lung cancer”. While trial designs of different indications correspond to trial processes, the drug A commonly corresponds to all of the processes.

On the other hand, in the clinical trial plan in this case, the indications are anamnesis, cancer type, or the like obtained by segmentalizing a disease and include an indication which is changed after a clinical trial process. For example, when a trial result in the phase 2 with the indication “lung cancer” is good, the indication may be expanded and include other cancer types such as stomach cancer in the phase 3.

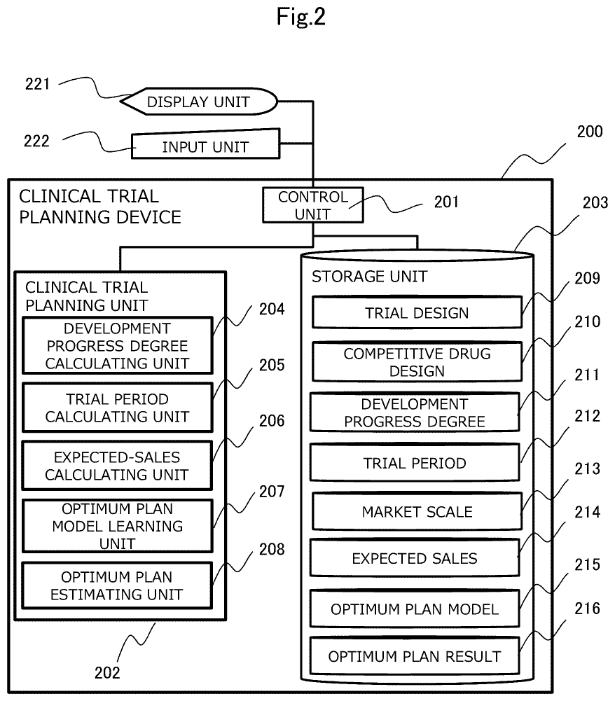

FIG. 2 is a block diagram illustrating a schematic configuration of a clinical trial planning device according to an embodiment of the present invention. A clinical trial planning device 200 has a control unit 201, a clinical trial planning unit 202, a storage unit 203, a display unit 221, and an input unit 222. The configuration can be realized by using an information processing device such as a general server, a personal computer, or the like. The configuration may be realized by a single device or realized in the cloud.

The control unit 201 is a processor executing a process in accordance with data stored in the storage unit 203 and a program stored in a memory (not illustrated) as a component of the clinical trial planning unit 202.

The clinical trial planning unit 202 has, for example, a semiconductor memory (not illustrated) storing a program which is executed by the control unit 201. The storage unit 203 has a storing device storing data or the like to which the control unit 201 refers. In the example of FIG. 2, in the clinical trial planning unit 202, a development progress degree calculating unit 204, a trial period calculating unit 205, an expected-sales calculating unit 206, an optimum plan model learning unit 207, and an optimum plan estimating unit 208 are stored.

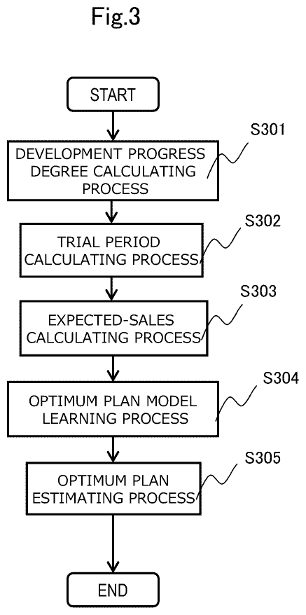

That is, in the following description, in practice, a process executed by each of the units is executed according to the program stored in the memory in the clinical trial planning unit 202 by the control unit 201. FIG. 3 illustrates the flow of the processes executed by the clinical trial planning unit 202.

In the storage unit 203, a trial design 209, a competitive drug design 210, a development progress degree 211, a trial period 212, a market scale 213, expected sales 214, an optimum plan model 215, and an optimum plan result 216 are stored.

The development progress degree calculating unit 204 obtains the trial design 209 and the competitive drug design 210 from the storage unit 203 and calculates, as the development progress degree of the competitive drug, the maximum value of the product of the response rate of the competitive design of the same indication and the development phase for all of trial designs in the trial design 209. As a result, the development progress degree 211 is stored in the storage unit 203.

The trial period calculating unit 205 obtains the trial design 209 from the storage unit 203, and calculates the trial period by using the number of samples for all of the trial designs in the trial design 209. As a result, the trial period 212 estimated as necessary for development of a drug of the one's company is stored in the storage unit 203.

The expected-sales calculating unit 206 reads the trial design 209, the market scale 213, and the development progress degree 211 from the storage unit 203, and calculates expected sales by using the trial design, the market scale, and the development progress degree for all of the trial designs in the trial design 209. As a result, the expected sales 214 is stored in the storage unit 203.

The optimum plan model learning unit 207 obtains the trial period 212 and the expected sales 214 from the storage unit 203, and learns an optimum plan model by reinforcement learning using the minimization of the trial period and the maximization of the expected sales as rewards. As a result, the optimum plan model 215 is stored in the storage unit 203.

The optimum plan estimating unit 208 obtains the trial period 212, the expected sales 214, and the optimum plan model 215 from the storage unit 203, and estimates a combination of optimum trial designs for each of the processes as an optimum plan. As a result, the optimum plan result 216 is stored in the storage unit 203.

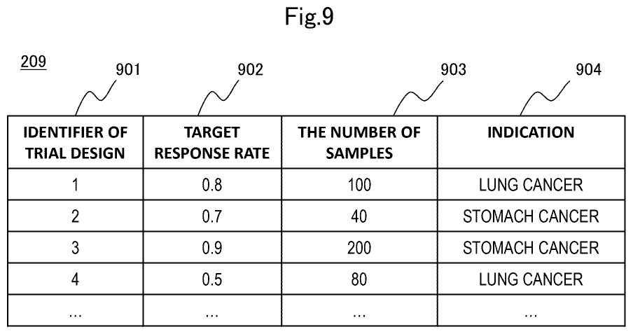

As the trial design 209, information of the identifier of a trial design which can be selected by one's company, a target response rate, the number of samples, and an indication is stored. FIG. 9 illustrates a data example of a trial design. Additionally, as a trial design, other end points such as a target hazard rate and a target survival duration, the necessity of an interim analysis, an end point of a control group, and the number of arms may be stored.

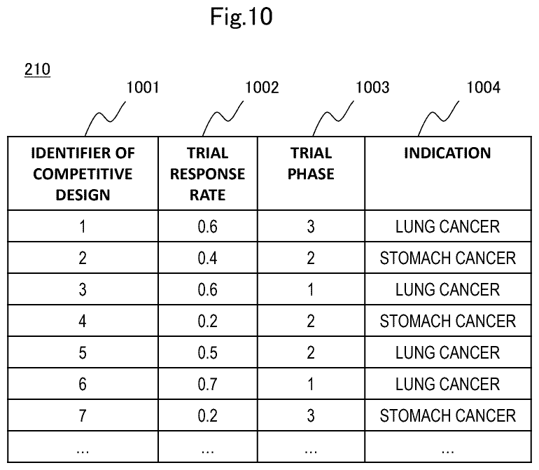

In the competitive drug design 210, by conducting a research on conference presentations of competitor companies and public clinical trial databases in Web sites, the identifier and a response rate of a competitive drug design, a development phase, and an indication are stored. FIG. 10 illustrates a data example of a competitive drug design. Additionally, in the competitive drug design, end points such as a target hazard rate and a target survival duration of a clinical trial of a competitive drug, the number of samples, the necessity of an interim analysis, an end point of a control group, and the number of arms may be stored.

As described above, the trial design 209 and the competitive drug design 210 have almost common specifications in the pharmaceutical industry. Consequently, by using data as it is from an arbitrary database or properly processing the data, it is easy to make the data of the trial design 209 and that of the competitive drug design 210 correspond to each other.

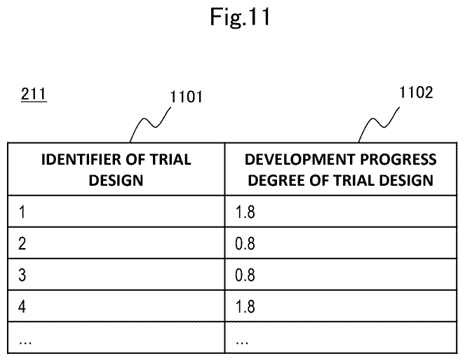

In the development progress degree 211, the identifier of a trial design and the development progress degree calculated by the development progress degree calculating unit 204 are stored. FIG. 11 illustrates a data example of the development progress degree.

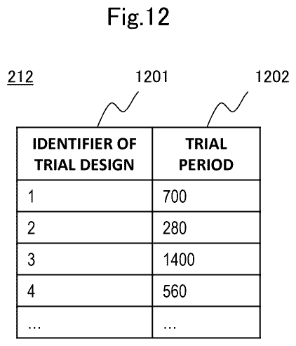

In the trial period 212, the identifier of a trial design, the identifier of a trial process, and a trial period calculated by the trial period calculating unit 205 are stored. FIG. 12 illustrates a data example of the trial period.

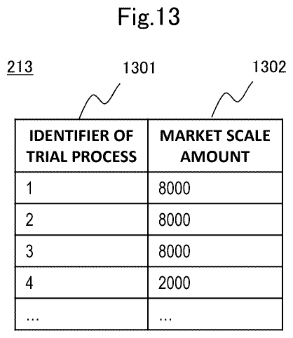

In the market scale 213, the identifier of each trial process and the market scale amount in the area of the trial process researched from epidemiological statistics are stored. FIG. 13 illustrates a data example of the market scale.

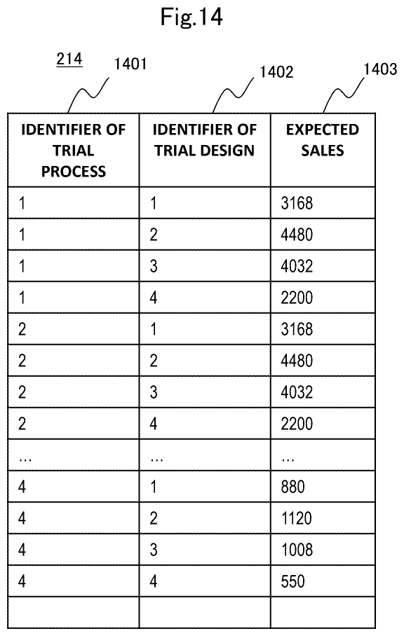

In the expected sales 214, the identifier of a trial design, the identifier of a trial process, and expected sales calculated by the expected-sales calculating unit 206 are stored. FIG. 14 illustrates a data example of expected sales.

In the optimum plan model 215, a model generated by the optimum plan model learning unit 207 is stored.

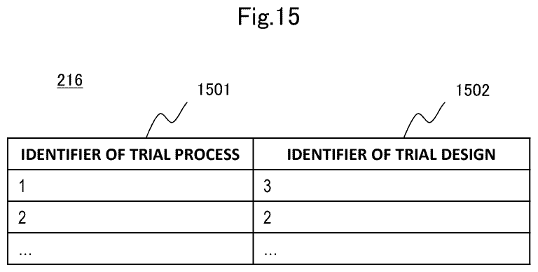

In the optimum plan result 216, a combination of each trial process and each trial design obtained by the optimum plan estimating unit is stored. FIG. 15 illustrates a data example of the optimum plan result.

In the display unit 221, three screens are displayed.

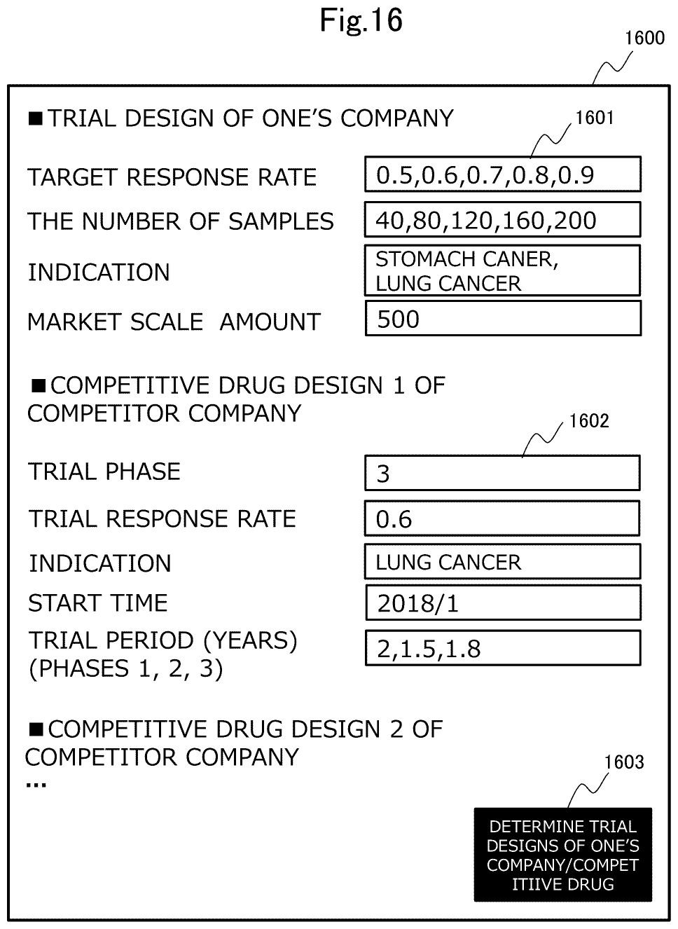

The first one is a one's company and a competitive drug trial design input screen. A screen for entering information of the identifier of a trial design which can be selected by one's company, a target response rate, the number of samples, and an indication, and a trial phase, a response rate, and an indication of a competitor company is displayed. In addition, as trial designs of one's company and a competitive drug, other end points such as a target hazard rate and a target survival duration, the necessity of an interim analysis, end points of a control group, the number of arms, and an eligibility criterion may be entered. In the input unit 222, those information is written and input. FIG. 16 illustrates an example of the one's company and competitive drug trial design input screen.

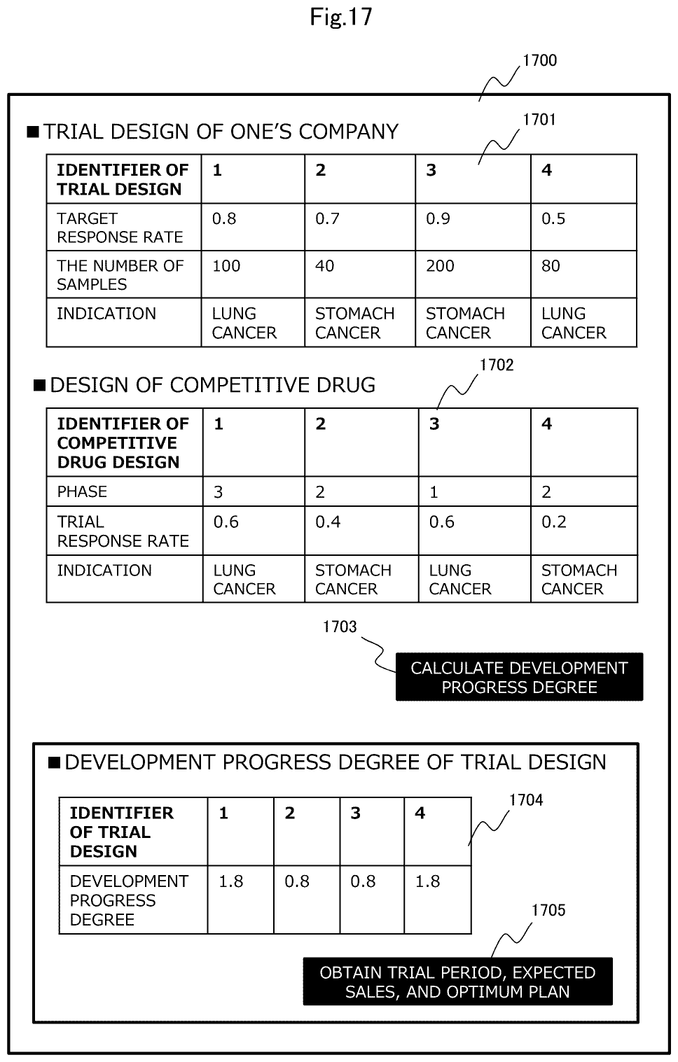

The second one is a development progress degree calculation screen. In this screen, the trial design of one's company and the trial design of the competitor company which are input from the one's company and competitive drug trial design input screen and, further, the development progress degree 211 obtained from the development progress degree calculating unit 204 are displayed. The input unit 222 gives an instruction to calculate the trial period and the expected sales and to estimate an optimum plan on the basis of the above-described information. FIG. 17 illustrates an example of the development progress degree calculation screen.

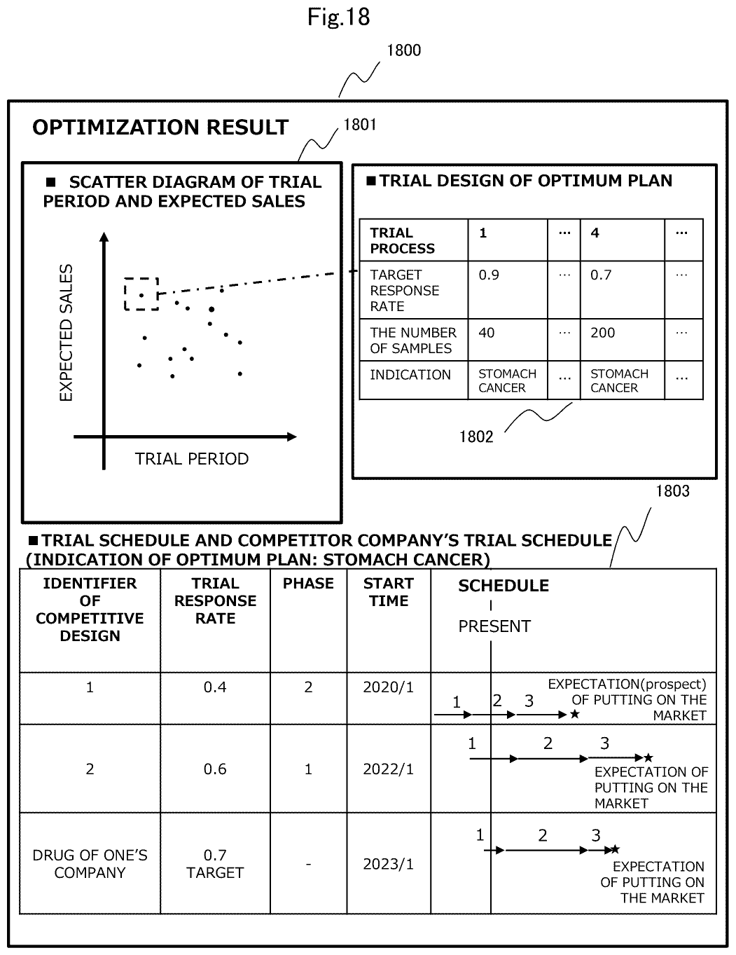

The third one is an optimum plan display screen. In this screen, an optimum plan is obtained from a trial design, a competitive drug design, and the development progress degree on the development progress degree calculation screen, and the trial period and the expected sales in the optimum plan, the trial design of each trial process, further, a schedule in the case of executing a clinical trial by the optimum plan, and a development schedule of the competitor company are displayed. The trial period and the expected sales of each trial plan calculated are displayed in a scatter diagram. When a plan in the scatter diagram is selected on the screen in the input unit 222, information of the trial design and the trial schedule is replaced by that of the selected plan. FIG. 18 illustrates an example of the optimum plan display screen.

The process flow will now be described.

FIG. 3 is a flowchart for explaining a clinical trial planning process executed by the clinical trial planning device according to the embodiment of the present invention. The clinical trial planning process of FIG. 3 corresponds to the process executed by the clinical trial planning unit 202 in FIG. 2.

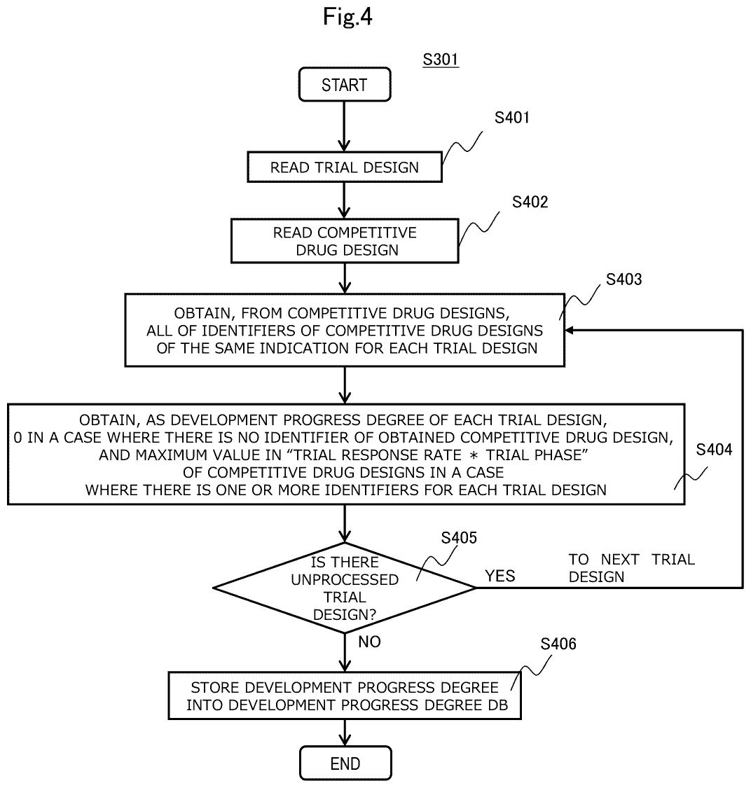

In step S301, the development progress degree calculating unit 204 obtains the trial design 209 (FIG. 9) and the competitive drug design 210 (FIG. 10) from the storage unit 203, and calculates, as the development progress degree, for example, the maximum value of the product between the response rate and the development phase of the competitive drug design of the same indication for all of trial designs in the trial design 209. The development progress degree expresses a progress situation of development of a competitive drug. In the execution mode of the embodiment, the development phases are five phases of 0 (before start), 1 (phase 1), 2 (phase 2), 3 (phase 3), and 4 (putting on the market). Other arbitrary numbers expressing development phases may be also used. As a result, the development progress degree 211 (FIG. 11) is stored in the storage unit 203. FIG. 4 illustrates a concrete process flow.

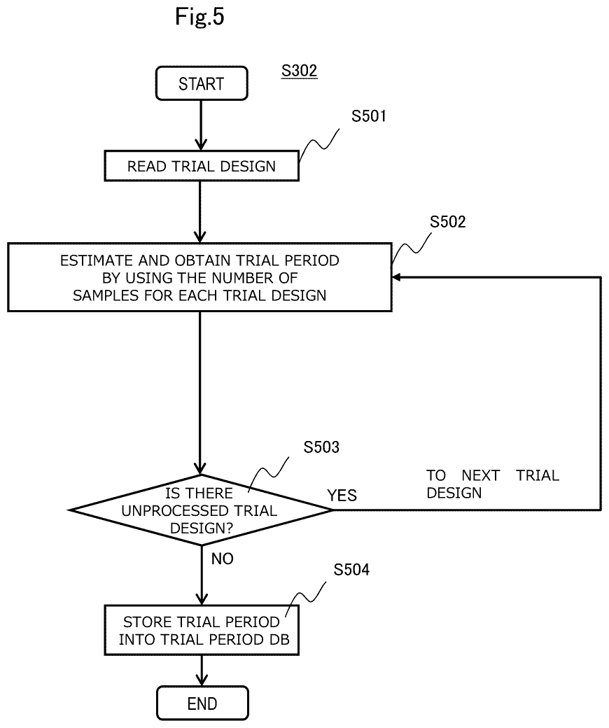

In step S302, the trial period calculating unit 205 obtains the trial design 209 (FIG. 9) from the storage unit 203, and estimates a trial period from the trial design for all of the trial designs in the trial design 209. The trial period expresses estimated development time of a drug of one's company. As a result, the trial period 212 (FIG. 12) is stored in the storage unit 203. FIG. 5 illustrates a concrete process flow.

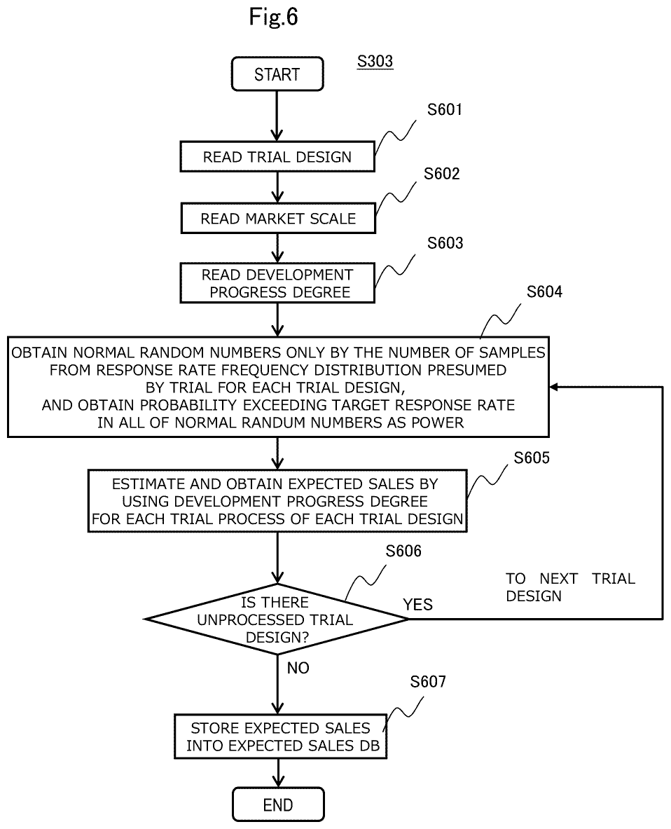

In step S303, the expected-sales calculating unit 206 reads the trial design 209 (FIG. 9), the market scale 213 (FIG. 13), and the development progress degree 211 (FIG. 11) from the storage unit 203, and calculates expected sales by using the trial design, the market scale, and the development progress degree for all of the trial designs in the trial design 209. As a result, the expected sales 214 (FIG. 14) is stored in the storage unit 203. FIG. 6 illustrates a concrete process flow.

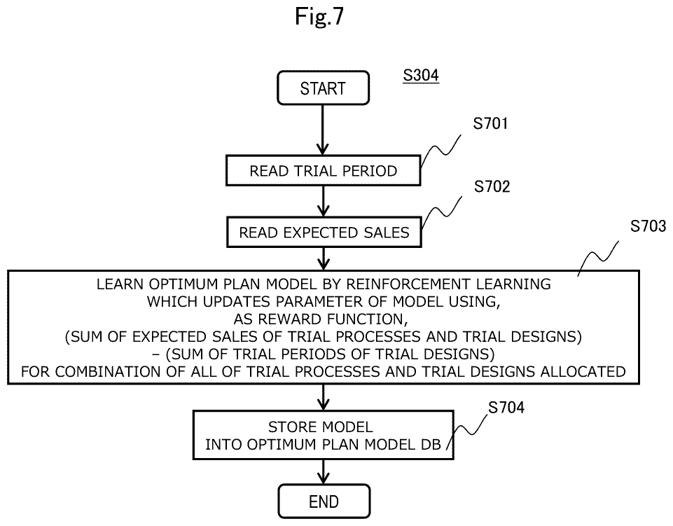

In step S304, the optimum plan estimating unit 208 obtains the trial period 212 (FIG. 12) and the expected sales 214 (FIG. 14) from the storage unit 203, and learns an optimum plan model by reinforcement learning using minimization of the trial period and maximization of the expected sales as rewards. As a result, the optimum plan model 215 is stored in the storage unit 203. FIG. 7 illustrates a concrete process flow.

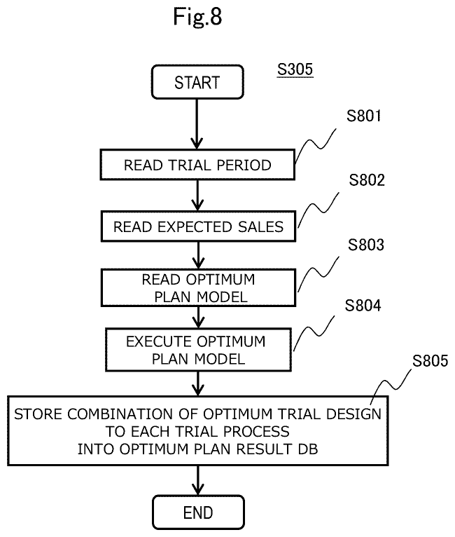

In step S305, the trial period 212 (FIG. 12), the expected sales 214 (FIG. 14), and the optimum plan model 215 are obtained from the storage unit 203, and a combination of optimum trial designs for each trial process is estimated as an optimum plan. As a result, the optimum plan result 216 is stored in the storage unit 203. FIG. 8 illustrates a concrete process flow. After that, the clinical trial planning unit 202 finishes the process.

FIG. 4 is a flowchart for explaining the development progress degree calculating process executed by the clinical trial planning device according to the embodiment of the present invention. The development progress degree calculating process of FIG. 4 corresponds to the development progress degree calculating unit 204 in FIG. 2 and the step S301 in FIG. 3.

In step S401, the trial design 209 (FIG. 1B and FIG. 9) is read from the storage unit 203.

In step S402, the competitive drug design 210 (FIG. 10) is read from the storage unit 203.

In step S403, all of identifiers of competitive drug designs of the same indication are obtained from the competitive drug designs for each of the trial designs. For example, in the data examples of FIGS. 9 and 10, the indication of the trial designs whose identifier is 1 is lung cancer, so that the identifiers 1, 3, 5, and 6 of the competitive drug designs whose indication is lung cancer are obtained.

In step S404, for each of the trial designs, in the case where there is no identifier of the competitive drug design obtained, 0 is derived. In the case where there is one or more identifiers of the competitive drug design obtained, the maximum value in the “trial response rate×trial phase” values of corresponding competitive drug designs is derived. This numerical value is obtained as the development progress degree of the competitive drug of each trial design. For example, in the trial design of the identifier 1, there are four identifiers 1, 3, 5, and 6 of the competitive drug designs of the same indication “lung cancer”. Consequently, the maximum value “1.8” among values of “trial response rate×phase” of “0.6×3=1.8”, “0.6×1=0.6”, “0.5×2=1.0”, and “0.7×1=0.7” is set as the development progress degree. “1.8” indicates the development progress degree of the competitive drug which is predicted to be productized earliest among competitive drugs for a drug which is to be developed by the development design of one's company.

In the above example, the development progress degree is evaluated by the trial response rate and the trial phase. This is an example. In place of the trial response rate, another index indicating an effect of a drug may be used. In place of the trial phase, an index indicating the progress degree of another development may be used. Other than the maximum value of the products of the response rate and the development phase of the competitive drug design, only the maximum value of the trial response rate or only the maximum value of the development phase may be used. In place of the maximum value, an average value or a weighted average value may be used. As weighting, a characteristic (company scale or technical power) of a development main body may be used. The development progress degree may be estimated by a predictor using machine learning.

In step S405, whether or not there is an unprocessed trial design or not in the trial design 209 is determined. When there is an unprocessed trial design, the development progress degree calculating unit 204 returns to step S403, and repeats the above-described process by using another unprocessed trial design.

In step S406, the development progress degree obtained in step S403 is stored as the development progress degree 211 (FIG. 11) in the storage unit 203. The development progress degree calculating unit 204 finishes the process.

FIG. 5 is a flowchart for explaining a trial period calculating process which is executed by the clinical trial planning device according to the embodiment of the present invention. The trial period calculating process of FIG. 5 corresponds to the trial period calculating unit 205 in FIG. 2 and step S302 in FIG. 3.

In step S501, the trial design 209 (FIG. 1B, FIG. 9) is read from the storage unit 203.

In step S502, a trial period is estimated by using the number of samples for each of the trial designs. For example, in the data example of FIG. 9, the number of samples of the trial design whose identifier is 1 is 100. In the case where it takes seven days for a clinical trial of one sample, the number of trial days is 100×7=700 (days). The required period per sample can be set in advance statistically or by knowledge of a skilled person. The above is an example. A trial period can be estimated by using other arbitrary calculating methods. For example, a trial period may be corrected by performing weighting or addition of the number of days by using the necessity or unnecessity or the number of times of an interim analysis. A trial period may be estimated by a predictor using machine learning.

In step S503, whether there is an unprocessed trial design in the trial design 209 or not is determined. When there is an unprocessed trial design, the development progress degree calculating unit 204 returns to step S502, and repeats the above-described process by using another unprocessed trial design.

In step S504, the trial period obtained in step S502 is stored as the trial period 212 in the storage unit 203. The trial period calculating unit 205 finishes the process.

FIG. 6 is a flowchart for explaining an expected-sales calculating process which is executed by the clinical trial planning device according to the embodiment of the present invention. The trial period calculating process in FIG. 6 corresponds to the trial period calculating unit 205 in FIG. 2 and step S303 in FIG. 3.

In step S601, the trial design 209 (FIGS. 1B and 9) is read from the storage unit 203.

In step S602, the market scale 213 (FIG. 13) is read from the storage unit 203.

In step S603, the development progress degree 211 (FIG. 11) is read from the storage unit 203.

In step S604, for each trial design, normal random numbers are obtained only by the number of samples from a normal distribution expressing the relation between the response rate and frequency of a drug assumed in a trial. The probability exceeding a target response rate in all of the normal random numbers is obtained as a power. For example, in the data example of FIG. 9, the number of samples of the trial design whose identifier is 1 is 100. When normal random numbers exceeding the target response rate are 90, the power is 0.9. The response rate and frequency of a drug assumed in a trial can be set in advance statistically on the basis of past data or by knowledge of a skilled person.

In step S605, the expected sale is obtained by estimation using the development progress degree for each of trial processes of each of trial designs. Alternatively, the expected sale may be estimated by using an arbitrary trial design and a development progress degree.

In the data examples of FIGS. 9, 11, and 13, the market scale amount of the trial process of the identifier 1 is 8000 in FIG. 13, the target response rate of the trial design of the identifier 1 is 0.8 in FIG. 9, the power is 0.9 in step S604, and the development progress degree is 1.8 in FIG. 11. When estimation is performed on assumption that expected sale=market scale amount×target response rate×power×(4−development progress degree)/4, the expected sale in the case of assigning the trial process of the identifier 1 to the trial design of the identifier 1 is 8000×0.8×0.9×(4−1.8)/4=3168. The last term “(4−development progress degree)/4)” corresponds to consideration of an amount of expected sale eroded by a competitive company, and is a penalty term. The term “(4−development progress degree)/4)” is an example, and the invention is not limited to the value as long as the development progress degree of other companies is reflected in a value.

In step S606, whether there is an unprocessed trial design in the trial design 209 or not is determined. In the case where there is an unprocessed trial design, the expected-sales calculating unit 206 returns to step S604 and repeats the above-described process by using another unprocessed trial design.

In step S607, the result obtained in step S605 is stored as the expected sales 214 (FIG. 14) in the storage unit 203. The expected-sales calculating unit 206 finishes the process.

FIG. 7 is a flowchart for explaining an optimum plan model learning process which is executed by the clinical trial calculating device according to the embodiment of the present invention. The optimum plan model learning process in FIG. 7 corresponds to the optimum plan model learning unit 207 in FIG. 2 and step S304 in FIG. 3.

In the optimum plan model learning process S304, a model which optimizes allocation of the trial design 209 (FIGS. 1B and 9) for each of the trial processes 101 of the clinical trial plan (FIG. 1A) of a targeted drug is learned.

In step S701, the trial period 212 (FIG. 12) is read from the storage unit 203.

In step S702, the expected sales 214 (FIG. 14) is read from the storage unit 203.

In step S703, the optimum plan model is learned by reinforcement learning which updates parameters of the model using, as a reward function, (total sum of expected sales corresponding to the combination of a trial process and a trial design)−(sum of trial period of trial design) for combinations of all of the trial processes and the trial designs allocated. That is, it is learned so that a larger reward is given to a clinical trial plan in which the expected sales is large and the trial period is short. The reward function is an example. The total sum of the expected sales, or the sum of the trial period, or both of the total sum of the expected sales and the sum of the trial period may be properly weighted. Ideally, the total sum of the expected sales is maximized, and the total of the trial period is minimized.

In step S704, the model is stored as the optimum plan model 215 into the storage unit 203. The optimum plan model learning unit 207 finishes the process.

Although the combinations of all of trial processes and trial designs are considered in the above-described step S703, the combinations may be limited as necessary.

For example, in the clinical trial plan illustrated in FIG. 1A, when a precondition is that a clinical trial on one indication is completed by phases 1 to 3, limitations are imposed that the trial design in the phase 1 corresponds to the trial processes 1 and 4, the trial design in the phase 2 corresponds to the trial processes 2 and 5, and the trial design in the phase 3 corresponds to the trial processes 3 and 6. By making a trial design including the same indication correspond to the trial processes 1 to 3 and making a trial design including the same indication correspond to the trial processes 4 to 6, a clinical trial related to one indication is completed by the clinical trial plan illustrated in FIG. 1A (refer to FIG. 1C).

FIG. 8 is a flowchart for explaining an optimum plan estimating process which is executed by the clinical trial planning device according to the embodiment of the present invention. The optimum plan estimating process in FIG. 8 corresponds to the optimum plan model learning unit 207 in FIG. 2 and step S305 in FIG. 3.

In step S801, the trial period 212 (FIG. 12) is read from the storage unit 203.

In step S802, the expected sales 214 (FIG. 14) is read from the storage unit 203.

In step S803, the optimum plan model 215 is read from the storage unit 203.

In step S804, each trial process and the trial period and expected sales of each trial designs are applied from the storage unit 203 to the optimum plan model 215, and optimization of the clinical trial plan is executed.

In step S805, a combination of an optimum trial design to each of the trial processes is stored in the optimum plan result 216.

By generating an optimum plan as described above, in the case of planning a clinical trial of high profitability in drug development of a cancer whose development is competitive, a problem such that a competitor company puts a drug of a similar indication on the market before one's company, differentiation from the drug of the competitor company cannot be made, and revenue cannot be increased can be solved.

Since a plan is made so as to put on the market a drug of an indication, which is differentiated from drugs of competitor companies according to the embodiment, a clinical trial planning method deciding a design of a clinical trial so as to optimize expected sales and a trial period calculated in consideration of the development progress degree calculated from an indication of a drug and a development phase of a competitor company is provided.

FIG. 9 is a diagram illustrating the data structure of a trial design held by the clinical trial planning device according to the embodiment of the present invention. For each trial design, an identifier 901 of the trial design, a target response rate 902, the number 903 of samples, and an indication 904 are stored. The data corresponds to the trial design 209 in FIG. 2. The trial design 209 is created by, for example, a person skilled in medicine manufacturing, and is recorded in the storage unit 203 in advance.

FIG. 10 is a diagram illustrating the data structure of a competitive drug design held by the clinical trial planning device according to the embodiment of the present invention. For a competitive drug being developed by competitor companies, an identifier 1001 of a competitive drug, a trial response rate 1002, a trial phase 1003, and an indication 1004 are stored. The data corresponds to the competitive drug design 210 in FIG. 2. The competitive drug design 210 is obtained from, for example, public pharmaceutical data or papers, and is recorded in the storage unit 203 in advance.

FIG. 11 is a diagram illustrating the data structure of a development progress degree held by the clinical trial planning device according to the embodiment of the present invention. For each trial design, an identifier 1101 of the trial design, and a development progress degree 1102 of the trial design are stored. The data corresponds to the development progress degree 211 in FIG. 2. The development progress degree 211 is calculated by the development progress degree calculating unit 204 and is recorded in the storage unit 203.

FIG. 12 is a diagram illustrating the data structure of a trial period held by the clinical trial planning device according to the embodiment of the present invention. For each trial design, an identifier 1201 of the trial design, and a trial period 1202 of the trial design are stored. The data corresponds to the trial period 212 in FIG. 2. The trial period 212 is calculated by the trial period calculating unit 205 and is recorded in the storage unit 203.

FIG. 13 is a diagram illustrating the data structure of a market scale held by the clinical trial planning device according to the embodiment of the present invention. For each trial process, an identifier 1301 of the trial process and a market scale amount 1302 (in arbitrary unit) are stored. The data corresponds to the market scale 213 in FIG. 2. The market scale 213 is determined, for example, on the basis of the number of patients in a certain area examined by epidemiology and statistics of statistical resources, papers, and databases which are open, and recorded in the storage unit 203 in advance. In the example of FIG. 1C, the market scale of the trial process 1 in which a trial is conducted in Japan to receive approval in Japan is the number of cancer patients in Japan. In the market scale amount, variations in the number of patients by indications may not be reflected.

FIG. 14 is a diagram illustrating the data structure of expected sales held by the clinical trial planning device according to the embodiment of the present invention. For a combination of each trial process and a trial design, an identifier 1401 of the trial process, an identifier 1402 of a trial design, and expected sales 1403 are stored. The data corresponds to the expected sales 214 in FIG. 2. The expected sales 214 is calculated by the expected-sales calculating unit 206 and is recorded in the storage unit 203.

FIG. 15 is a diagram illustrating the data structure of an optimum plan result held by the clinical trial planning device according to the embodiment of the present invention. A combination of a trial process of an optimum plan and a trial design is stored as an identifier 1501 of the trial process and an identifier 1502 of the trial design. The data corresponds to the optimum plan result 216 in FIG. 2. The optimum plan result 216 is estimated by the optimum plan model 215, output, and recorded in the storage unit 203.

FIG. 16 is an explanatory diagram illustrating a display example of a one's company and competitive drug trial design input screen by the clinical trial planning device according to the embodiment of the present invention. A one's company and competitive drug trial design input screen 1600 has a one's company's trial design input field 1601, a competitor company's competitive drug design input field 1602, and a button 1603 for entering them.

First, the user enters a target response rate, the number of samples, an indication, and a market scale amount which can be selected by the one's company into the input fields 1601 of the trial design of the one's company. Subsequently, the trial phase, a trial response rate, an indication, start timing, and a trial period of each trial phase of a drug of the competitor company are entered in the input fields 1602 of the competitive drug design of the competitor company.

FIG. 17 is an explanatory diagram illustrating a display example of a development progress degree calculation screen by the clinical trial planning device according to the embodiment of the present invention. A development progress degree calculation screen 1700 has a trial design 1701 of one's company, a design 1702 of a competitive drug, a button 1703 for calculating a development progress degree, a development progress degree 1704 of the trial design, and a button 1705 for obtaining a trial period, expected sales, and an optimum plan.

The user first checks the trial design 1701 of the one's company and the design 1702 of the competitive drug displayed, and clicks the button 1703 for calculating the development progress degree to obtain the development progress degree. Subsequently, the user checks the development progress degree 1704 of the trial design and clicks the button 1705 for obtaining a trial period, expected sales, and an optimum plan to optimize the clinical trial plan.

FIG. 18 is an explanatory diagram illustrating a display example of an optimum plan display screen by the clinical trial planning device according to the embodiment of the present invention. An optimum plan display screen 1800 has a scatter diagram 1801 of a trial period and expected sales, an optimum plan information 1802, and a trial schedule and competitor situation 1803.

The user checks the scatter diagram 1801 of the trial period and expected sales, information 1802 of an optimum plan, and the trial schedule and competitor situation 1803 displayed. By clicking a plot on the scatter diagram 1801 of the trial period and expected sales, a plan to be selected can be switched, and display of the information 1802 of an optimum plan, the trial schedule, and the competitor situation 1803 changes.

According to the above embodiment, the conventional problem such that, at the time of developing a plan of a clinical trial, competition of an indication of a development drug occurs and it becomes difficult to assure profit depending on start/end of a clinical trial of a competitive drug and depending on a patient indication is solved. Planning of a clinical trial of the same indication as that of competitor companies, in which development is made before other companies so that profit is assured can be made.

According to the embodiment, an efficient clinical trial can be realized. Consequently, the invention can contribute to realization of sustainable society by decreasing consumption energy, reducing carbon emission, and preventing global warming.

### REFERENCE SIGNS LIST

200: clinical trial planning device
201: control unit
202: memory
203: storage unit
221: display unit
222: input unit

What is claimed is:

1. A clinical trial planning device comprising a clinical trial planning unit, wherein
the clinical trial planning unit
includes a development progress degree calculating unit calculating a development progress degree of a second drug of which patient indication corresponds to that of a first drug for which a clinical trial is to be planned, on the basis of a competitive drug design indicating a situation of a clinical trial related to the second drug, and
generates a clinical trial plan obtained by optimizing a period of a clinical trial of the first drug and expected sales of the first drug on the basis of the development progress degree.

2. The clinical trial planning device according to claim 1, wherein
the clinical trial plan is a combination of a plurality of trial designs related to the first drug.

3. The clinical trial planning device according to claim 2, wherein
the trial design defines the patient indication, the number of samples, and a target response rate.

4. The clinical trial planning device according to claim 3, wherein
the clinical trial planning unit
comprises a trial period calculating unit estimating a period of a clinical trial of the first drug on the basis of the number of samples for each of the trial designs and storing it into a trial period database.

5. The clinical trial planning device according to claim 3, wherein
the clinical trial planning unit
comprises an expected-sales calculating unit estimating expected sales of the first drug on the basis of a market scale and the target response rate of the first drug and the development progress degree for each of the trial designs, and storing it into an expected-sales database.

6. The clinical trial planning device according to claim 5, wherein
the expected-sales calculating unit
makes the market scale and the target response rate of the first drug act in the direction of increasing the expected sales of the first drug, and makes the development progress degree act in the direction of decreasing the expected sales of the first drug.

7. The clinical trial planning device according to claim 5, wherein
the expected-sales calculating unit obtains normal random numbers only by the number of samples from a normal distribution expressing a response rate and frequency of the first drug assumed by a clinical trial, sets a probability exceeding the target response rate in all of normal random numbers as a power, and estimates expected sales of the first drug on the basis of the power.

8. The clinical trial planning device according to claim 3, wherein
the competitive drug design is stored in a competitive drug design database which defines the patient indication, a trial response rate, and a trial phase.

9. The clinical trial planning device according to claim 8, wherein
a development progress calculating unit extracts all of second drugs whose patient indication corresponds to that of the first drug from the competitive drug design database, calculates the development progress degree on the basis of the response rate of the second drug extracted and a trial phase, and stores the development progress degree into a development progress degree database.

10. The clinical trial planning device according to claim 2, wherein
the clinical trial planning unit comprises an optimum plan estimating unit evaluating the optimization on the basis of a mathematical formula using both a period of a clinical trial of the first drug and expected sales of the first drug.

11. The clinical trial planning device according to claim 10, wherein
the optimization is performed by a reinforcement learning method using a reward function with the mathematical formula being as the reward function.

12. The clinical trial planning device according to claim 10, wherein
the optimization is performed so as to minimize a total sum of a period of a clinical trial of the first drug and maximize a total sum of expected sales of the first drug with respect to the trial design included in the clinical trial plan.

13. A clinical trial planning method which is executed by an information processing device having a control unit, a clinical trial planning unit, a storage unit, a display unit, and an input unit, wherein
the clinical trial planning unit executes:
a first step of reading a plurality of trial designs defining a patient indication and the number of samples, related to a first drug;
a second step of reading a plurality of competitive drug designs defining a patient indication and a trial phase, related to a second drug;
a third step of specifying a competitive drug design having the same patient indication from the competitive drug designs for each of the trial designs;
a fourth step of calculating a development progress degree on the basis of the trial phase of the competitive drug design specified for each of the trial designs;
a fifth step of calculating a trial period on the basis of the number of samples for each of the trial designs;
a sixth step of calculating expected sales on the basis of a market scale and the development progress degree for each of the trial designs; and
a seventh step of generating a clinical trial plan made by a combination of the trial designs by using a reward function having the trial period and the expected sales as variables, for a combination of the trial designs allocated to trial processes of the number which is preliminarily determined.

14. The clinical trial planning method according to claim 13, wherein
in the seventh step,
an optimum plan model generated by reinforcement learning using miniaturization of the total sum of the trial period and maximization of the total sum of the expected sales as rewards is used.

15. The clinical trial planning method according to claim 13, wherein
a target response rate is further defined in the trial design read in the first step,
a trial response rate is further defined in the competitive drug design read in the second step,
the development progress degree is further calculated by using the trial response rate in the fourth step, and
the expected sales is further calculated by using the target response rate in the sixth step.
